(19) United States
(12) Patent Application Publication
(10) Pub. No.: US 2007/0213676 A1
(43) Pub. Date: Sep. 13, 2007

(54) Cleaning apparatus and method

(76) Inventor: Uigaese Mac Popoalii, Tacoma, WA (US)

Correspondence Address: UIGAESE MAC POPOALII, 13801 PARK AVE S., TACOMA, WA, 98444, US

(21) Appl. No.: 11/713,158

(22) Filed: Mar. 3, 2007

Related U.S. Application Data
(60) Provisional application No. 60/781,005, filed on Mar. 10, 2006.
(60) Provisional application No. 60/799,436, filed on May 10, 2006.

(51) Int. Cl. A61M 31/00

(52) U.S. Cl. 604/279

(57) ABSTRACT

The present invention provides an apparatus for cleaning a body cavity, the apparatus having a hollow tube, a pump shaft, a pump head, and a nozzle, the pump shaft being located within the hollow tube and slidably passing through a self-sealing hole in a closed end, the pump head being removably attached to the shaft, and the nozzle being removably attached to an open end of the tube.

### PRIORITY

The applicant claims priority from a Provisional Patent Application filed on Mar. 10, 2006 under Application No. 60/781,005, and from a Provisional Patent Application filed on May 10, 2006 under Application No. 60/799,436.

### FEDERALLY SPONSORED RESEARCH

None.

### FIELD OF THE INVENTION

The present invention generally relates to an apparatus to irrigate the vaginal cavity with either water or a cleaning solution and to provide sexual stimulation.

### BACKGROUND OF THE INVENTION

In the area of female personal hygiene, many devices exist to irrigate the vaginal cavity. Many women find this desirable to address health concerns, eliminate odors, to increase general hygiene, or as a way to remove some semen from the vagina as a contraceptive aid. In addition, several devices are available to provide sexual stimulation. Only a few devices exist to provide both irrigation and stimulation.

For example, U.S. Pat. No. 6,589,216 discloses a vaginal douche applicator designed to facilitate the flow of the cleansing fluid into and out of the vagina. However, this device provides no pump or other mechanism to cause fluid to flow into the cavity, but only a valve to limit the flow. U.S. Pat. No. 2,792,003 discloses a device to distribute a medicament next to the cervix. The device incorporates a plunger mechanism, but the portion of the device to be inserted into the cavity is not customizable by the user. Neither of these devices is intended to provide sexual stimulation to the user. US Patent Application 2006/0084895 discloses a device both to cleanse the vagina and to provide a massage. However, the means of dispensing the cleansing fluid into the vagina is inconvenient. Other prior art devices have similar disadvantages. None of the prior art devices are adapted to conveniently irrigate the vagina with two separate fluids.

Accordingly, there is a need for a device with a convenient and controllable method of dispensing two types of fluid, nozzles that are customizable to the individual user's size or preference, and that can be used to provide sexual stimulation. The present invention fulfills this and other needs.

### SUMMARY OF THE INVENTION

The present invention is composed of a two hollow tubes, each tube having an open end and a closed end and being attached to a center portion or locking mechanism at the closed end; a pump shaft located within each tube and passing through the locking mechanism; a pump head attached to the pump shaft; and a nozzle attached to the open end of one of the hollow tubes.

In use, the nozzle and pump head are removed and one of the hollow tubes is detached from the locking mechanism. The tube is filled with a fluid and replaced onto the pump shaft, but not attached to the locking mechanism. The pump head and nozzle are reattached to the filled tube. The filled tube is inserted into the vagina and the empty tube, which is connected to the locking mechanism and pump shaft, is pulled back and forth to pump the fluid out of the nozzle of the filled tube. In the preferred embodiment, one tube may be filled with a first fluid, and the other tube may be filled with a second fluid, so that the user may first clean the vagina, and then rinse with water, for example.

The above summary of the present invention is not intended to represent each embodiment, or every aspect, of the present invention. Additional features and benefits of the present invention are apparent from the detailed description and figures set forth below.

### DESCRIPTION OF THE FIGURES

FIG. 1 is a cross-sectional side view of the device and its component parts.

FIG. 2 is a perspective view of the closed end.

FIG. 3 is a side view of an alternate embodiment of the pump shaft.

FIG. 4 depicts the parts of one embodiment of the pump head.

FIG. 5 depicts the parts of another embodiment of the pump head.

FIG. 6 is a cross-sectional side view of an alternate embodiment in which the nozzle has been inverted for storage.

FIG. 7 is a perspective view of a nozzle and hose adapter.

FIG. 8 is a side view of an inflation nozzle and sleeve.

FIG. 9 is a perspective view of an alternate nozzle.

FIG. 10 is a perspective view of a discharge valve.

While the invention is susceptible to various modifications and alternative forms, specific embodiments are shown by way of example in the drawings and are described in detail herein. It should be understood, however, that the invention is not intended to be limited to the particular forms disclosed. Rather, the invention is to cover all modifications, equivalents, and alternatives falling within the spirit and scope of the invention as described.

### DETAILED DESCRIPTION OF THE ILLUSTRATED EMBODIMENTS

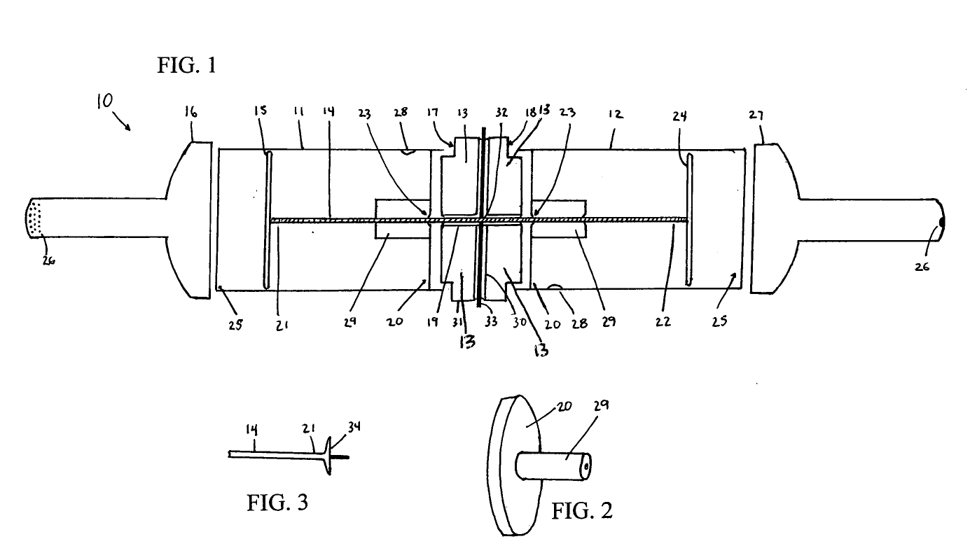

Referring to FIG. 1, a cleaning apparatus (10) is shown having a first hollow tube (11) and a second hollow tube (12), a center portion or locking mechanism (13), a pump shaft (14), at least one pump head (15), and at least one nozzle (16). The locking mechanism (13) has a first side (17), a second side (18) directly opposite the first side (17), and a hollow shaft (19) between the first side (17) and the second side (18). The first tube (11) is removably attached to the first side (17) at a closed end (20), and the second tube (12) is similarly removably attached to the second side (18) at a closed end (20). The first side (17), second side (18), and the closed end (20) of each tube may be threaded so as to allow the tubes to be attached by screwing them onto the first and second sides.

The pump shaft (14) has a first end (21) and a second end (22) and is located substantially in the center of each tube (11, 12) and the hollow shaft (19) of the locking mechanism (13) such that the first end (21) of the pump shaft is located within the first tube (11) and the second end (22) is within the second tube (12). The pump shaft (14) passes through a self-sealing hole (23) in the closed end (20) of each tube (11, 12). The pump head (15) is removably attached to the first end (21) of the pump shaft, and a second pump head (24) may be removably attached to the second end (22) in the preferred embodiment. A nozzle (16) is removably attached to an open end (25) of the first tube (11), the open end (25) being located opposite the closed end (20), and the nozzle (16) having an exit hole or holes (26) through which a fluid may be pumped out. In the preferred embodiment, a second nozzle (27) with holes (26) is similarly attached to an open end (25) of the second tube (12). The nozzles and the open end of each tube may be threaded to so as to allow the nozzles to be attached by screwing them onto the open ends. The tube, pump shaft, and other parts may take any appropriate shape.

A discharge valve (28) is positioned near the closed end (20) of the tube (11) to allow the device to be drained of fluid. In the preferred embodiment, the pump shaft (14) is rigid and may be formed of plastic or another suitable material. In some embodiments, the pump shaft (14) passes through the hole (23) and into a stabilizing portion (29), which has a diameter smaller than that of the tube but greater than that of the pump shaft, and is provided to ensure that the pump shaft remains substantially in the center of the tube. The stabilizing portion may also have a rubber o-ring or other means to prevent the fluid from leaking out of the hollow tube. The tube may be formed of a rigid plastic such as polypropylene or any other suitable material.

In the embodiment depicted in FIG. 1, a first hole (30) extends through a diameter of the locking mechanism (13) from an outer surface (31) of the locking mechanism (13) through the hollow shaft (19). A second hole (32) is located in the pump shaft (14). A bar (33) may be removably inserted into the first hole (30) in the locking mechanism and through the second hole (32) in the pump shaft. When the bar (33) is inserted, the pump shaft will not move.

In an alternate embodiment, the pump shaft is immovably attached to the center portion or locking mechanism, and no bar, hole, or hollow shaft is necessary. Thus, a first pump shaft may attach to the first side of the center portion and a second pump shaft may attach to the second side of the center portion. In another embodiment, the bar may be U-shaped and there may be multiple holes in the locking mechanism and pump shaft. In yet another embodiment, a bar or bars are located within a hole in the locking mechanism extending from the outer surface to the hollow shaft, with a first bar end adjacent to the hollow shaft and the pump shaft therein and a second bar end protruding from the outer surface of the locking mechanism. When pressure is exerted on the second bar end, the first bar end will come into contact with the pump shaft, immobilizing the pump shaft. In another alternate embodiment, the locking mechanism incorporates a spring and is configured such that it will move from a first position in which the bar does not immobilize the pump shaft to a second position in which the pump shaft is immobilized when pressure is exerted on the end of the bar, as with the mechanism in a retractable ballpoint pen.

To use the device, the nozzle (16) and pump head (15) are removed from one of the tubes (11) and the closed end (20) of the tube is detached from the locking mechanism (13). The tube (11) is then filled with a fluid through the open end (25) and replaced onto the pump shaft (14), but not attached to the locking mechanism (13). The nozzle (16) and pump head (15) are then reattached. The filled tube (11) is inserted into the vagina and the empty tube (12), which is connected to the locking mechanism (13) and pump shaft (14), is pulled back and forth to pump the fluid out of the nozzle (16) of the filled tube (11). In addition, the user may fill the first tube (11) with a first fluid (for example, a cleaning solution, spermicidal fluid, medicine, or an edible substance) and the second tube (12) with a second fluid (for example, water for rinsing), and the user may conveniently pump first one fluid, and then the other.

In some embodiments, the nozzles may be long and flexible, and have a smaller diameter than the diameter of the hollow tube, in order to more easily fit inside the body cavity intended to be cleaned. For vaginal cleaning, the nozzles may be shaped like flexible objects for sexual stimulation available in the prior art. In the preferred embodiment, the nozzle would be formed of latex, silicone, plastic, rubber, or another suitable material, such as would be familiar in the art of items to be inserted in the vaginal cavity for hygiene or pleasure. In the embodiment employing two nozzles, one nozzle may be flexible and the other rigid to be used as the user prefers. In an alternate embodiment, the nozzles may be irremovably attached to the tubes.

Referring to FIG. 2, an alternate embodiment is shown in which the closed end (20) and stabilizing portion (29) is a single piece separate from the tube (11), but is threaded or otherwise shaped so that it may be securely attached to the tube (11). A rubber disk or o-ring may be provided to ensure that no fluid can escape through the closed end.

In the preferred embodiment, the exit holes (26) of the nozzle (16) are of sufficiently small diameter to minimize the amount of water or other fluid that may drip out under the force of gravity when the apparatus is inverted. A diameter of approximately 2 mm has been found to be appropriate, but other diameters may be suitable.

In the preferred embodiment as shown in FIG. 3, the first or second end (21, 22)) of the pump shaft (14) is formed to provide a shelf (34) for the pump head (15) to rest against.

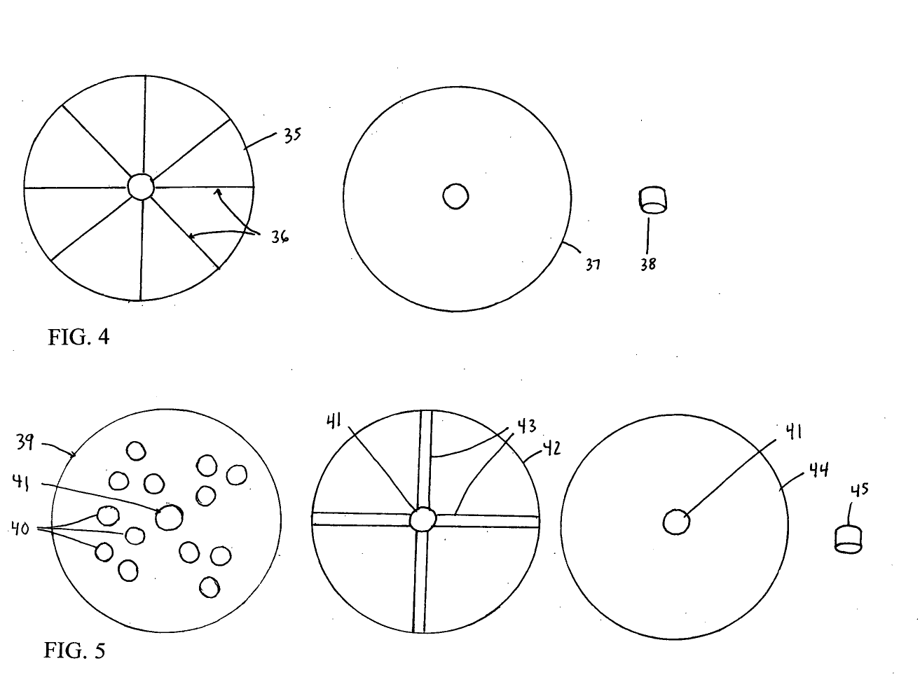

Referring to FIG. 4, an embodiment of the pump head (15) is shown, configured so that the fluid will freely pass through the pump head (15) when it is moving in one direction, but when moving in the opposite direction, the pump head will push the fluid. Thus, the pump head (15) may comprise a first disk (35), with spokes (36) extending from an edge of the disk to a center of the disk, and a second disk (37) formed of a flexible material such as rubber. The first disk (35) is placed on the pump shaft (14), with the second disk (37) placed over the first disk (35), and a pin (38) is screwed onto the end of the pump shaft (14) to secure the disks in place. The first disk may be of metal, plastic, or any other suitable material, and should be of such a diameter that it touches the tube to minimize fluid that passes around the edge of the first disk, and yet is still easily moved back and forth within the tube. The edge may have a rubber coating.

When moving the pump head (15) in a first direction toward the open end, the second disk (37) will lie flat against the spokes (36) of the first disk (35) preventing fluid from passing between the spokes (36) and thereby pushing the fluid out through the nozzle. When moving in a second opposite direction away from the open end, the second disk (37) will bend away from the spokes (36), allowing the fluid will pass between the spokes (36) and through the pump head (15).

Referring to FIG. 5, the preferred embodiment of the pump head (15) is shown, comprising a first disk (39), which is substantially solid but has a configuration of holes (40) aligned along diameters of the disk and a center hole (41); a second disk (42), which is also substantially solid but has one or more openings (43) and a center hole (41); a third disk (44) which is formed of a flexible material such as rubber and has a center hole (41); and a pin (45). In use, the first disk (39) is placed first onto the pump shaft (14), the second disk (42) is placed on top of the first disk (39) and may be twisted so that a different configuration of holes (40) is exposed through the openings (43) or that no holes are exposed, the third disk (44) is placed on top of the second disk (42), and the pin (45) is placed on top of the third disk and screwed onto the pump shaft (14) to secure the disks in place, with the pump shaft (14) passing through the center hole (41) of each disk. As described above, the third disk (44) will flex as the pump head (15) is moved so that the pump head (15) will push the fluid when it is moving in one direction, but the fluid will pass through the pump head (15) when it is moving in the opposite direction.

In the preferred embodiment, the third disk (44) will only flex when pressure is exerted on the pump shaft so that the third disk (44) will remain flat against the second disk (42) when the apparatus is inverted. Also in the preferred embodiment, the second and third disks (42, 44) are of a slightly smaller diameter than the first disk (39) so that they do not contact the tube (11), while the first disk (39) is of such diameter that it contacts the tube (11) to minimize the amount of fluid that passes around the disk, but is not too great to make it difficult to move the disk back and forth when in use.

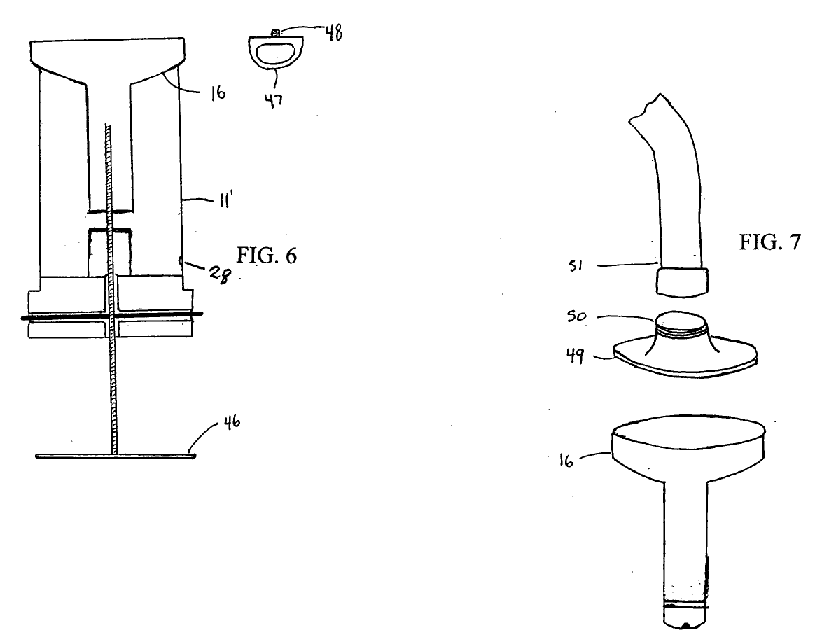

Referring to FIG. 6, an alternate embodiment is shown having a single hollow tube (11′). In this embodiment, there is a handle (46) at the end of the pump shaft. In this and other embodiments, the nozzles (16, 27) may be of such a shape that they can be inverted and placed inside an empty tube (11, 12). The nozzles may therefore have a nozzle tip that can be removed. Further, the pump heads may be removed from the pump shaft. A storage adapter (47) may be provided that can be inserted securely into the nozzle (16) after it has been inverted and placed inside the empty tube. The storage adapter (47) has a threaded stem (48), onto which the pump head may be placed for storage. In this and other embodiments, the nozzles and the open end of the tube may be shaped so that the nozzle may be fitted securely onto the tube either when in use or when inverted for storage without the need to thread either the nozzle or the open end.

Referring to FIG. 7, a hose adapter (49) is depicted that may be inserted into the nozzle (16). The adapter has a threaded portion (50) of appropriate diameter that it may be screwed onto a standard hose (51) for attaching to a shower head or faucet.

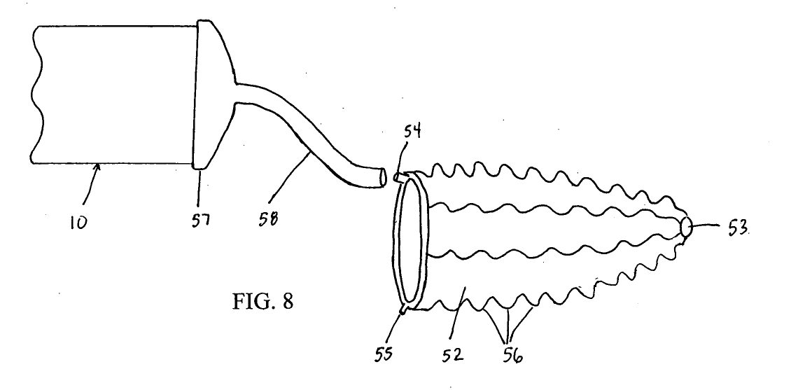

Referring to FIG. 8, a sleeve (52) is provided that may be placed over the nozzle (16) alone or over the nozzle (16) and the hollow tube (11). The sleeve (52) includes a sleeve exit hole (53) at one end for the passage of the fluid being pumped out of the nozzle (16). The exterior of the sleeve (52) may be of any suitable material, but usually rubber, silicone, or latex, and it may be of various sizes. The sleeve (52) may be inflated with air, water, or another suitable substance via a valve (54). Any appropriate type of valve familiar in the prior art may be used. Some embodiments may have a first inflation valve (54) through which the sleeve is inflated and a second deflation valve (55) to be used when the user wishes to deflate the sleeve. In use, the sleeve may be inflated either before or after inserting the device into the vagina so that the inflated sleeve fits the contours of the vagina. In some embodiments, the exterior surface of the sleeve may have multiple soft bristles or protrusions (56) to aid in massaging or cleaning the cavity.

Also referring to FIG. 8, an inflation nozzle (57) may be provided and used in place of the nozzle (16) so that the apparatus (10) itself may be used to inflate the sleeve (52). The inflation nozzle (57) may attach directly to the valve (54) of the sleeve (52), or the inflation nozzle may be attached to a flexible hose (58), which in turn is attached to the valve (54). In an alternate embodiment, the sleeve may have a self-contained pump mechanism. When the apparatus is used in this manner as an air pump, the pump head depicted in FIG. 5 and described in paragraph 29 above is configured as an air pump available in the prior art, such as by twisting the second disk so that there are no holes exposed or by placing the third flexible disk between the first and second disks.

The device as a whole and the nozzle may be formed to resemble a penis, and the device may be used for sexual stimulation in addition to its hygienic uses.

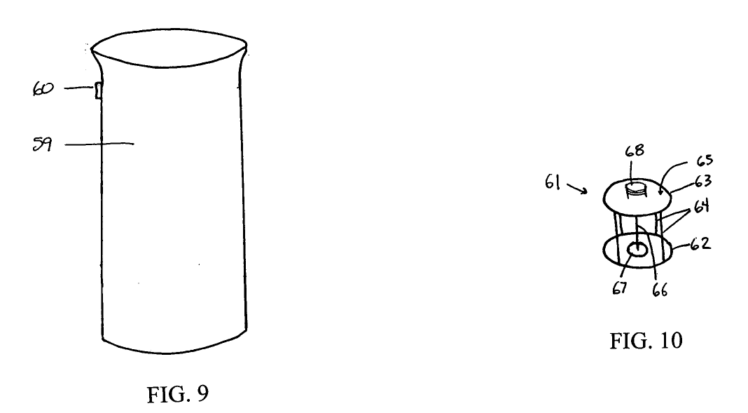

Referring to FIG. 9, a male nozzle (59) is depicted, which may be attached to the device in place of the nozzle (16) to provide stimulation to males. The male nozzle (59) is essentially a hollow tube with a valve (60) in order to equalize the pressure inside the tube and would be used as a standard penis pump available in the prior art. When the apparatus is used in this manner, the pump head (15) depicted in FIG. 5 and described in paragraph 29 above may be inverted so that the flexible rubber disk is closest to the closed end and the other disks are twisted so that only one hole is exposed to enable the apparatus to pump air out of the tube and male nozzle when in use.

Referring to FIG. 10, an alternate discharge valve (61) is depicted to facilitate use of the device to pump air out of the male nozzle (59). The valve (61) is comprised of a first valve portion (62) and a second valve portion (63), the first valve portion (62) having a threaded aperture of substantially the same size as the discharge hole (28) in the tube (11, 12) that may be screwed into the discharge hole (28). The first and second valve portions are connected by a plurality of ribs (64). The second valve portion (63) is solid and has an exterior side (65) and an interior side. A rigid stem (66) extends from the second valve portion (63) toward the first valve portion (62), with a flexible disk (67) at an end of the stem (66), such that the flexible disk (67) covers the aperture and discharge hole (28). When air is flowing out of the discharge hole (28), the flexible disk (67) will flex away from the hole and allow air to escape. When no air flows out of the discharge hole (28), the flexible disk (67) rests against the aperture and discharge hole (28), sealing the tube (11). The exterior side (65) of the second valve portion (63) has threading (68) such that it may be screwed into the discharge hole to seal the hole completely.

I claim:

1. A cleaning apparatus comprising:
a first hollow tube, a pump shaft, a first pump head, and a first nozzle, said first hollow tube having a first open end and a first closed end, said pump shaft being located within said first hollow tube and slidably passing through a self-sealing hole in said first closed end, said first pump head being removably attached to a first end of said pump shaft located within said first hollow tube, and said first nozzle being removably attached to said first open end.

2. The cleaning apparatus of claim 1, further comprising:
a center portion having a first side, and a second side opposite said first side, the first closed end of said first hollow tube being removably attached to said first side, said pump shaft attached to said center portion; and
a second hollow tube having a second open end and a second closed end, said second closed end being removably attached to said second side, said pump shaft passing through a second self-sealing hole in said second closed end.

3. The cleaning apparatus of claim 2, further comprising a discharge valve located near said closed end of said first hollow tube.

4. The cleaning apparatus of claim 2, further comprising:
a second pump head removably attached to a second end of said pump shaft located within said second hollow tube, and a second nozzle removably attached to said second open end of said second hollow tube.

5. The cleaning apparatus of claim 2, in which said first nozzle is formed to resemble a human penis.

6. The cleaning apparatus of claim 1, further comprising:
a locking mechanism having a first side, a second side opposite said first side, and a hollow shaft extending between said first side and said second side, the first closed end of said first hollow tube being removably attached to said first side, said pump shaft passing through said hollow shaft;
a second hollow tube having a second open end and a second closed end, said second closed end being removably attached to said second side, said pump shaft passing through a second self-sealing hole in said second closed end; and
a locking hole located in said locking mechanism, extending from an outer surface of said locking mechanism to said hollow shaft, and a locking bar inserted into said locking hole such that a first end of said locking bar comes into contact with said pump shaft when pressure is exerted on a second end of said locking bar.

7. The cleaning apparatus of claim 6, further comprising means to hold said locking bar alternately in a first position in which the locking bar is not in contact with the pump shaft and in a second position in which the locking bar is in contact with the pump shaft.

8. The cleaning apparatus of claim 1, further comprising:
a locking mechanism having a first side, a second side opposite said first side, and a hollow shaft extending between said first side and said second side, the first closed end of said first hollow tube being removably attached to said first side, said pump shaft passing through said hollow shaft;
a second hollow tube having a second open end and a second closed end, said second closed end being removably attached to said second side, said pump shaft passing through a second self-sealing hole in said second closed end; and
a locking hole located in said locking mechanism, extending from an outer surface of said locking mechanism to said hollow shaft, and a locking bar inserted into said locking hole such that a first end of said locking bar enters a hole in said pump shaft when pressure is exerted on a second end of said locking bar.

9. The cleaning apparatus of claim 1, in which said pump head comprises means to restrict the passage of a fluid through said pump head.

10. The cleaning apparatus of claim 1, in which said pump head comprises:
a first disk having spokes extending from an edge to a center of the first disk, a second disk placed over said first disk, and a pin placed onto said pump shaft over said second disk to secure said first and second disk in place;
said second disk being flexible such that said second disk lies flat against said spokes when said pump head is moving in a first direction toward said open end, thereby preventing passage of a fluid between said spokes, and such that said second disk bends away from said spokes when said pump head is moving in a second opposite direction away from the open end, allowing the fluid to pass between the spokes.

11. The cleaning apparatus of claim 1, in which said pump head comprises:
a first disk having holes arranged along diameters of the first disk and a center hole, a second disk having one or more openings, a third disk placed over said second disk, and a pin placed onto said pump shaft over said third disk to secure said first, second, and third disks in place;
said second disk being rotatable such that various configurations of holes on said first disk are exposed through said openings in said second disk; and
said third disk being flexible such that it lies flat against said second disk when said pump head is moving in a first direction toward said open end, thereby preventing passage of a fluid through said exposed holes, and such that said third disk bends away from said second disk when said pump head is moving in a second opposite direction away from the open end, allowing the fluid to pass through said exposed holes.

12. The cleaning apparatus of claim 1, in which said first nozzle may be inverted and inserted in to said first hollow tube.

13. The cleaning apparatus of claim 12, in which said first nozzle includes a removable nozzle tip.

14. The cleaning apparatus of claim 1, further comprising a sleeve with an exit hole that is removably placed over said nozzle to increase the size of said nozzle.

15. The cleaning apparatus of claim 1, further comprising an inflatable sleeve with an exit hole that is removably placed over said nozzle.

16. The cleaning apparatus of claim 15, further comprising an inflation nozzle which may be used in place of said nozzle to inflate said sleeve using said apparatus.

17. The cleaning apparatus of claim 14 in which an outer surface of said sleeve has a plurality of protrusions.

18. The cleaning apparatus of claim 1, further comprising an adapter inserted into said nozzle such that said nozzle may be connected to a standard hose.

19. The cleaning apparatus of claim 1 in which said nozzle is in the shape of a hollow cylinder and in which said pump head is configured to pump air out of said cylindrical nozzle.

20. The cleaning apparatus of claim 19, further comprising a one-way valve located in said tube near said closed end.

21. The cleaning apparatus of claim 20 in which said one-way valve comprises: a first valve portion with a discharge hole, said first valve portion being connected to a second valve portion by one or more ribs; a rigid stem extending from said second portion to said discharge hole, said stem having a flexible disk of such size that it covers said discharge hole.

22. A cleaning apparatus comprising:
a first hollow tube, a pump shaft, a first pump head, and a first nozzle, said first hollow tube having a first open end and a first closed end, said pump shaft being located within said first hollow tube and slidably passing through a self-sealing hole in said first closed end, said first pump head being removably attached to a first end of said pump shaft located within said first hollow tube, and said first nozzle being removably attached to said first open end;
a center portion having a first side, and a second side opposite said first side, the first closed end of said first hollow tube being removably attached to said first side, said pump shaft attached to said center portion;
a second hollow tube, a second pump head, and a second nozzle, said second hollow tube having a second open end and a second closed end, said second closed end being removably attached to said second side, said pump shaft slidably passing through a second self-sealing hole in said second closed end, said second pump head being removably attached to a second end of said pump shaft located within said second hollow tube, and said second nozzle being removably attached to said second open end.

23. The cleaning apparatus of claim 22, in which said first and second pump heads comprise:
a first disk having holes arranged along diameters of the first disk and a center hole, a second disk having one or more openings, a third disk placed over said second disk, and a pin placed onto said pump shaft over said third disk to secure said first, second, and third disks in place;
said second disk being rotatable such that various configurations of holes on said first disk are exposed through said openings in said second disk; and
said third disk being flexible such that it lies flat against said second disk when said pump head is moving in a first direction toward said open end, thereby preventing passage of a fluid through said exposed holes, and such that said third disk bends away from said second disk when said pump head is moving in a second opposite direction away from the open end, allowing the fluid to pass through said exposed holes.

24. The cleaning apparatus of claim 23 in which said first and second ends of said pump shaft widen to form a support for said first and second pump heads.

25. The cleaning apparatus of claim 23, further comprising a first stabilizing portion around said pump shaft and attached to said first closed end in substantially the center of said closed end and a second stabilizing portion around said pump shaft attached to said second closed end in substantially the center of said closed end.

26. The cleaning apparatus of claim 22, further comprising an inflatable sleeve with an exit hole that is removably placed over said first or said second nozzle.

27. The cleaning apparatus of claim 23, in which an outer surface of said sleeve has a plurality of protrusions.

28. The cleaning apparatus of claim 23, in which said sleeve further comprises a self-contained pump.

29. A method of cleaning a body cavity, comprising the steps of:
filling a first hollow tube of a cleaning apparatus with a fluid, attaching a first pump head to a pump shaft located within said first hollow tube, and attaching a first nozzle to said first hollow tube;
inserting said first nozzle into said body cavity and pumping said fluid out of said first hollow tube.

30. The method of claim 29, further comprising the steps of:
filling a second hollow tube of said cleaning apparatus with a second fluid, attaching a second pump head to said pump shaft also located within said second hollow tube, and attaching a second nozzle to said second hollow tube;
inserting said second nozzle into said body cavity and pumping said second fluid out of said second hollow tube.

31. The cleaning apparatus of claim 1, further comprising
a center portion having a first side, and a second side opposite said first side, the first closed end of said first hollow tube being removably attached to said first side, said pump shaft attached to the first side of said center portion, and a second pump shaft attached to the second side of said center portion;
a second hollow tube having a second open end and a second closed end, said second closed end being removably attached to said second side, said second pump shaft slidably passing through a second self-sealing hole in said second closed end;
a second pump head removably attached to an end of said second pump shaft located within said second hollow tube; and
a second nozzle removably attached to said second open end.
